(19) United States
(12) Patent Application Publication
(10) Pub. No.: US 2025/0134871 A1
(43) Pub. Date: May 1, 2025

(54) UPR MODULATORS TO TREAT HEARING LOSS

(71) Applicant: JACARANDA BIOSCIENCES, INC., San francisco, CA (US)

(72) Inventor: Russell Dahl, Saint John, IN (US)

(21) Appl. No.: 18/719,364

(22) PCT Filed: Dec. 16, 2022
(86) PCT No.: PCT/US2022/053106

§ 371 Date: Jun. 13, 2024

Related U.S. Application Data
(60) Provisional application No. 63/290,296, filed on Dec. 16, 2021.

(51) Int. Cl. A61K 31/44; A61K 31/167

(52) U.S. Cl. CPC A61K 31/44; A61K 31/167

(57) ABSTRACT

Provided herein are compounds of Formula (I), pharmaceutical compositions thereof, and methods of their use for treating, preventing, or ameliorating hearing loss.

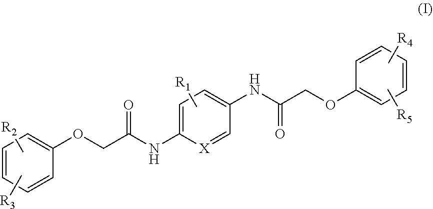

### RELATED APPLICATIONS

This application claims the benefit of U.S. Provisional Application Ser. No. 63/290,296, filed Dec. 16, 2021, the entire teachings of which are incorporated herein by reference.

### FIELD OF INVENTION

Provided herein are novel compounds, pharmaceutical compositions thereof, and methods of use for treating hearing loss (HL).

### BACKGROUND

Noise-induced hearing loss (NIHL) is a profound public health problem, affecting over 40 million Americans and causing the loss of 4 million disability-adjusted life years worldwide from occupational exposure annually. HL is also a significant risk factor for dementia, underscoring the expanded morbidity of this disorder. 27% of NIHL is from acute sound exposure, whereas 23% occurs from sub-acute or chronic exposure to sound, suggesting that interventions that target the mechanisms that underlie acute or sub-acute HL can make an immediate and transformative clinical impact for this large and inadequately treated patient population.

Hearing loss can also be mediated by drug exposure. This includes the following classes of drugs, aminoglycosides (e.g. tobramycin and amikacin), anti-cancer drugs (cisplatin) and loop diuretics (furosemide) and phosphodiesterase 5 inhibitors (tadalafil) (Prescrire Int. 2014, 23, 290-294). Individuals who must take these medications for critical medical needs should not have to suffer such a morbid side-effect from the treatment.

Endoplasmic reticulum (ER) stress and the unfolded protein response (UPR) are key drivers of NIHL pathophysiology (Li, et al., J. Clin. Invest. 2018, 128, 5150-5162; Rouse, et al., Sci. Rep. 2020, 10, 18063). In addition to NIHL, other modes of hearing loss, such as side effects from drugs such as cisplatin, can lead to hearing loss (Kros & Steyger, Cold Spring Harb. Perspect. Med. 2019, 9, a033548). Both NIHL and drug toxicity may be mediated through the UPR, which is a component of ER responses. The ER is the site of nearly all synthesis, folding, and maturation of transmembrane and secreted proteins before transit through the Golgi for additional post-translational modification and delivery to different cellular compartments. The UPR is a series of highly conserved intra-ER processes that assess in real time the capacity of the ER for protein folding and processing. These signaling cascades can be activated to increase the capacity of the cell to handle an extra load of protein synthesis, to downregulate the rate of protein synthesis to immediately ameliorate the excess load of unfolded proteins or, if the load is too great, to activate pathways leading to cell apoptosis. Three main parallel signaling pathways constitute the UPR, which are labeled by the initial protein in the pathway that senses change in protein synthesis regulation: PERK (double stranded RNA-activated protein kinase (PKR)-like ER kinase), IRE1 (inositol requiring enzyme 1), and ATF6 (activating transcription factor 6). PERK activation upregulates transcription of CHOP, a transcription factor that controls genes involved in apoptosis in addition to the UPR. IRE1 activation affects ER homeostasis through sXBP1, leading to upregulation of molecular chaperones (that aid in folding proteins) and proteins involved in ERAD (endoplasmic reticulum associated protein degradation). Similarly, activation of ATF6 upregulates target genes that mitigate ER stress, including BiP (binding immunoglobulin protein; GRP78), an ER chaperone that translocates newly synthesized proteins across the ER membrane, enhances protein folding, directs mis-folded proteins to ERAD and helps regulate Ca²⁺ homeostasis. Thus, the UPR comprises a wide range of interacting pathways that facilitate homeostatic control over many ER processes but can lead to cell death if excessively perturbed.

Modulation of the UPR has been shown to affect multiple forms of hearing loss, including NIHL (Li, et al., J. Clin. Invest. 2018, 128, 5150-5162; Rouse, et al., Sci. Rep. 2020, 10, 18063). Modulating the protein CHOP (transcription factor C/EBP homologous protein) and proteins in its signaling pathway (including PERK and eiF2B) can participate in the pathway that inhibits multiple forms of hearing loss (Li, et al., J. Clin. Invest. 2018, 128, 5150-5162). The link between hearing loss and the UPR has been demonstrated by genetic inhibition of the key UPR pathways (Li, et al., J. Clin. Invest. 2018, 128, 5150-5162). Additionally, it has been shown that cochlear synaptopathy (diminished wave I amplitude and loss of synapses between the cochlear nerve and inner hair cells) is prevented by ISRIB (integrated stress response inhibitor), a compound that mediates the UPR (Summers, et al., J. Mol. Biol. 1987, 196, 175-198). ISRIB can modulate the UPR in cell lines, cochlear hair cells and can prevent hearing loss in mice from noise exposure (permanent threshold shift; Li, et al., J. Clin. Invest. 2018, 128, 5150-5162) and can prevent cochlear synatopathy after a transient threshold shift (Rouse, et al., Sci. Rep. 2020, 10, 18063).

### SUMMARY

This disclosure provides compositions and methods comprising compounds of Formula I.

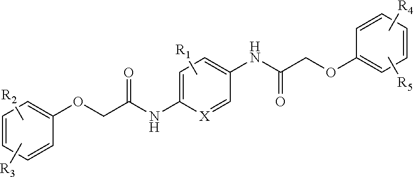

or a pharmaceutically acceptable salt, solvate, or hydrate thereof which are useful in modulation of the UPR as demonstrated by their ability to reduce the ER stress marker CHOP in thapsigargin-treated HEK 293 cells. The invention also comprises pharmaceutical compositions comprising a therapeutically effective amount of compound of Formula I, or a pharmaceutically acceptable salt, solvate, or hydrate thereof. The invention disclosed herein is also directed to a method of treating a hearing loss condition. The invention disclosed herein is also directed to a method of treating age-related hearing loss, noise-induced hearing loss, genetic or inherited hearing loss, hearing loss due to ototoxic exposure, disease-induced hearing loss, trauma-induced hearing loss, and cochlear synaptopathy.

### DETAILED DESCRIPTION

### Definitions

To facilitate understanding of the disclosure set forth herein, a number of terms are defined below.

Generally, the nomenclature used herein and the laboratory procedures in organic chemistry, medicinal chemistry, and pharmacology described herein are those well known and commonly employed in the art. Unless defined otherwise, all technical and scientific terms used herein generally have the same meaning as commonly understood by one of ordinary skill in the art to which this disclosure belongs.

The term “subject” refers to an animal, including, but not limited to, a primate (e.g., human), cow, pig, sheep, goat, horse, dog, cat, rabbit, rat, or mouse. The terms “subject” and “patient” are used interchangeably herein in reference, for example, to a mammalian subject. In one embodiment, the subject is a human.

The terms “treat,” “treating,” and “treatment” are meant to include alleviating a disorder, disease, or condition, or alleviating or reducing one or more symptoms of the disorder, disease, or condition; or alleviating the cause(s) of the disorder, disease, or condition itself. In the context of treating hearing loss, “treat,” “treating,” and “treatment” are meant to include slowing the progression of hearing loss or reversing hearing loss.

The term “therapeutically effective amount” is meant to include the amount of a compound that, when administered, to alleviate to some extent, one or more symptoms of the disorder, disease, or condition being treated. The term “therapeutically effective amount” also refers to the amount of a compound that is sufficient to elicit the biological or medical response of a biological molecule (e.g., a protein, enzyme, RNA, or DNA), cell, tissue, system, animal, or human, which is being sought by a researcher, veterinarian, medical doctor, or clinician.

The term “pharmaceutically acceptable carrier,” “pharmaceutically acceptable excipient,” “physiologically acceptable carrier,” or “physiologically acceptable excipient” refers to a pharmaceutically acceptable material, composition, or vehicle, such as a liquid or solid filler, diluent, solvent, or encapsulating material. In one embodiment, each component is “pharmaceutically acceptable” in the sense of being compatible with other ingredients of a pharmaceutical formulation, and suitable for use in contact with the tissue or organ of humans and animals without excessive toxicity, irritation, allergic response, immunogenicity, or other problems or complications, commensurate with a reasonable benefit/risk ratio. See, Remington: The Science and Practice of Pharmacy, 21st Edition, Lippincott Williams & Wilkins: Philadelphia, PA, 2005; Handbook of Pharmaceutical Excipients, 7th Edition, Rowe et al., Eds., The Pharmaceutical Press and the American Pharmaceutical Association: 2012; Handbook of Pharmaceutical Additives, 3rd Edition, Ash and Ash Eds., Gower Publishing Company: 2007; and Pharmaceutical Preformulation and Formulation, 2nd Edition, Gibson Ed., CRC Press LLC: Boca Raton, FL, 2009.

The term “alkyl” refers to a linear or branched saturated monovalent

hydrocarbon radical. The term “alkyl” also encompasses both linear and branched alkyl, unless otherwise specified. In certain embodiments, the alkyl is a linear saturated monovalent hydrocarbon radical that has 1 to 20 (C₁₋₂₀), 1 to 15 (C₁₋₁₅), 1 to 10 (C₁₋₁₀), or 1 to 6 (C₁₋₆) carbon atoms, or branched saturated monovalent hydrocarbon radical of 3 to 20 (C₃₋₂₀), 3 to 15 (C₃₋₁₅), 3 to 10 (C₃₋₁₀), or 3 to 6 (C₃₋₆) carbon atoms. As used herein, linear C₁₋₆ and branched C₃₋₆ alkyl groups are also referred as “lower alkyl.” Examples of alkyl groups include, but are not limited to, methyl, ethyl, propyl (including all isomeric forms), n-propyl, isopropyl, butyl (including all isomeric forms), n-butyl, isobutyl, sec-butyl, t-butyl, pentyl (including all isomeric forms), and hexyl (including all isomeric forms). For example, C₁₋₆ alkyl refers to a linear saturated monovalent hydrocarbon radical of 1 to 6 carbon atoms or a branched saturated monovalent hydrocarbon radical of 3 to 6 carbon atoms. In certain embodiments, the alkyl is optionally substituted with one or more substituents Q as described herein.

The term “alkenyl” refers to a linear or branched monovalent hydrocarbon radical, which contains one or more, in one embodiment, one, two, three, four, or five, in another embodiment, one, carbon-carbon double bond(s). In certain embodiments, the alkenyl is optionally substituted with one or more substituents Q as described herein. The term “alkenyl” also embraces radicals having “cis” and “trans” configurations, or alternatively, “Z” and “E” configurations, as appreciated by those of ordinary skill in the art. As used herein, the term “alkenyl” encompasses both linear and branched alkenyl, unless otherwise specified. For example, C₂₋₆ alkenyl refers to a linear unsaturated monovalent hydrocarbon radical of 2 to 6 carbon atoms or a branched unsaturated monovalent hydrocarbon radical of 3 to 6 carbon atoms. In certain embodiments, the alkenyl is a linear monovalent hydrocarbon radical of 2 to 20 (C₂₋₂₀), 2 to 15 (C₂₋₁₅), 2 to 10 (C₂₋₁₀), or 2 to 6 (C₂₋₆) carbon atoms, or a branched monovalent hydrocarbon radical of 3 to 20 (C₃₋₂₀), 3 to 15 (C₃₋₁₅), 3 to 10 (C₃₋₁₀), or 3 to 6 (C₃₋₆) carbon atoms. Examples of alkenyl groups include, but are not limited to, ethenyl, propen-1-yl, propen-2-yl, allyl, butenyl, and 4-methylbutenyl.

The term “alkynyl” refers to a linear or branched monovalent hydrocarbon radical, which contains one or more, in one embodiment, one, two, three, four, or five, in another embodiment, one, carbon-carbon triple bond(s). In certain embodiments, the alkynyl is optionally substituted with one or more substituents Q as described herein. The term “alkynyl” also encompasses both linear and branched alkynyl, unless otherwise specified. In certain embodiments, the alkynyl is a linear monovalent hydrocarbon radical of 2 to 20 (C₂₋₂₀), 2 to 15 (C₂₋₁₅), 2 to 10 (C₂₋₁₀), or 2 to 6 (C₂₋₆) carbon atoms, or a branched monovalent hydrocarbon radical of 3 to 20 (C₃₋₂₀), 3 to 15 (C₃₋₁₅), 3 to 10 (C₃₋₁₀), or 3 to 6 (C₃₋₆) carbon atoms. Examples of alkynyl groups include, but are not limited to, ethynyl (—C≡CH) and propargyl (—CH₂C≡CH). For example, C₂₋₆ alkynyl refers to a linear unsaturated monovalent hydrocarbon radical of 2 to 6 carbon atoms or a branched unsaturated monovalent hydrocarbon radical of 3 to 6 carbon atoms.

The term “halogen”, “halide” or “halo” refers to fluorine, chlorine, bromine, and/or iodine.

When a group is substituent, is a group such as an alkyl, alkenyl, alkynyl, cycloalkyl, aryl, aralkyl, heteroaryl, and heterocyclyl group, may be substituted with one or more substituents Q, each of which is independently selected from, e.g., (a) oxo (═O), cyano (—CN), halo, and nitro (—NO₂); (b) C₁₋₆ alkyl, C₂₋₆ alkenyl, C₂₋₆ alkynyl, C₃₋₁₀ cycloalkyl, C₆₋₁₄ aryl, C₇₋₁₅ aralkyl, heteroaryl, and heterocyclyl, each of which is further optionally substituted with one or more, in one embodiment, one, two, three, four, or five, substituents Q^{a}; and (c) —C(O)R^{a}, —C(O)OR^{a}, —C(O)NR^{b}R^{c}, —C(NR^{a})NR^{b}R^{c}, —OR^{a}, —OC(O)R^{a}, —OC(O)OR^{a}, —OC(O)NR^{b}R^{c}, —OC(═NR^{a})NR^{b}R^{c}, —OS(O)R^{a}, —OS(O)₂R^{a}, —OS(O)NR^{b}R^{c}, —OS(O)₂NR^{b}R^{c}, —NR^{b}R^{c}, —NR^{a}C(O)R^{d}, —NR^{a}C(O)OR^{d}, —NR^{a}C(O)NR^{b}R^{c}, —NR^{a}C(═NR^{d})NR^{b}R^{c}, —NR^{a}S(O)R₂, —NR^{a}S(O)₂R^{d}, —NR^{a}S(O)NR^{b}R^{c}, —NR^{a}S(O)₂NR^{b}R^{c}, —P(O)R^{a}R^{d}, —P(O)(OR^{a})R^{d},
—P(O)(OR^{a})(OR^{d}), —SR^{a}, —S(O)R^{a}, —S(O)₂R^{a}, —S(O)NR^{b}R^{c}, and —S(O)₂NR^{b}R^{c}, wherein each R^{a}, R^{b}, R^{c}, and R^{d} are independently (i) hydrogen; (ii) C₁₋₆ alkyl, C₂₋₆ alkenyl, C₂₋₆ alkynyl, C₃₋₁₀ cycloalkyl, C₆₋₁₄ aryl, C₇₋₁₅ aralkyl, heteroaryl, or heterocyclyl, each of which is optionally substituted with one or more, in one embodiment, one, two, three, or four, substituents Q^{a}; or (iii) R^{b} and R^{c} together with the N atom to which they are attached form heteroaryl or heterocyclyl, each of which is optionally substituted with one or more, in one embodiment, one, two, three, or four, substituents Q^{a}. Alternatively, R^{a}, R^{b}, R^{c}, and R^{d} are independently H or C₁₋₆alkyl.

In one embodiment, each substituent Q^{a} is independently selected from the group consisting of (a) oxo, cyano, halo, and nitro; and (b) C₁₋₆ alkyl, C₂₋₆ alkenyl, C₂₋₆ alkynyl, C₃₋₁₀ cycloalkyl, C₆₋₁₄ aryl, C₇₋₁₅ aralkyl, heteroaryl, and heterocyclyl; and (c) —C(O)R^{e}, —C(O)OR^{e},
—C(O)NR^{f}R^{g}, —C(NR^{e})NR^{f}R^{g}, —OR^{e}, —OC(O)R^{e}, —OC(O)OR^{e}, —OC(O)NR^{f}R^{g}, —OC(═NR^{e})NR^{f}R^{g}, —OS(O)R^{e}, —OS(O)₂R^{e}, —OS(O)NR^{f}R^{g}, —OS(O)₂NR^{f}R^{g}, —NR^{f}R^{g}, —NR^{e}C(O)R^{h}, —NR^{e}C(O)OR^{h}, —NR^{e}C(O)NR^{f}R^{g}, —NR^{e}C(═NR^{h})NR^{f}R^{g}, —NR^{e}S(O)R^{h}, —NR^{e}S(O)₂R^{h}, —NR^{e}S(O)NR^{e}R^{g}, —NR^{e}S(O)₂NR^{f}R^{g}, —P(O)R^{e}R^{h}, —P(O)(OR^{e})R^{h}, —P(O)(OR^{e})(OR^{h}), —SR^{e}, —S(O)Re, —S(O)₂R^{e}, —S(O)NR^{f}R^{g}, and —S(O)₂NR^{f}R^{g}; wherein each R^{e}, R^{f}, R^{g}, and R^{h} is independently (i) hydrogen, C₁₋₆ alkyl, C₂₋₆ alkenyl, C₂₋₆ alkynyl, C₃₋₁₀ cycloalkyl, C₆₋₁₄ aryl, C₇₋₁₅ aralkyl, heteroaryl, or heterocyclyl; or (ii) R^{f} and R^{g} together with the N atom to which they are attached form heteroaryl or heterocyclyl. Alternatively, each R^{e}, R^{f}, R^{g}, and R^{h} is independently hydrogen or C₁₋₆ alkyl.

Another embodiment of the invention is a compound disclosed herein or a pharmaceutically acceptable salt, solvate or hydrate thereof, in which one or more hydrogen atoms is replaced with deuterium. The deuterium enrichment at any one of the sites where hydrogen has been replaced by deuterium is at least 50%, 75%, 85%, 90%, 95%, 98% or 99%. Deuterium enrichment is a mole percent and is obtained by dividing the number of compounds with deuterium enrichment at the site of enrichment with the number of compounds having hydrogen or deuterium at the site of enrichment.

The term “solvate” refers to a complex or aggregate formed by one or more molecules of a solute, e.g., a compound provided herein, and one or more molecules of a solvent, which present in a stoichiometric or non-stoichiometric amount. Suitable solvents include, but are not limited to, water, methanol, ethanol, n-propanol, isopropanol, and acetic acid. In certain embodiments, the solvent is pharmaceutically acceptable. In one embodiment, the complex or aggregate is in a crystalline form. In another embodiment, the complex or aggregate is in a noncrystalline form. Where the solvent is water, the solvate is a hydrate. Examples of hydrates include, but are not limited to, a hemihydrate, monohydrate, dihydrate, trihydrate, tetrahydrate, and pentahydrate.

### Compounds

Provided herein is a compound of Formula I:

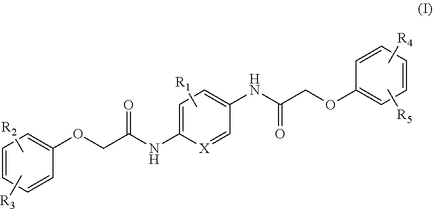

or a pharmaceutically acceptable salt, solvate, or hydrate thereof; wherein:
R¹-R⁵ are independently selected from hydrogen, cyano, nitro, or halo; C₁₋₆ unsubstituted alkyl, C₂₋₆ unsubstituted alkenyl, or C₂₋₆ unsubstituted alkynyl; —CF₃, —O—(C₁-C₄ alkyl), —O—CF₃, —OH, —NH (C₁-C₄ alkyl)₂ and —N(C₁-C₄ alkyl)₂; and
X is N or CH.

In another embodiment, the compound is of formula II:

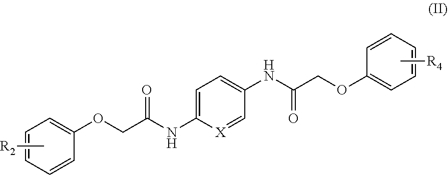

or a pharmaceutically acceptable salt, solvate, or hydrate thereof; wherein:
R¹ and R² are independently selected from hydrogen, cyano, nitro, or halo; C₁₋₆ unsubstituted alkyl, C₂₋₆ unsubstituted alkenyl, or C₂₋₆ unsubstituted alkynyl; —CF₃, —O—(C₁-C₄ alkyl), —O—CF₃, —OH, —NH(C₁-C₄ alkyl)₂ and —N(C₁-C₄ alkyl)₂; and
X is N or CH.

In another embodiment, the compound is of Formula I or II, with the proviso that:
when X═CH, R¹-R⁵ are not all simultaneously H;
when X═CH and R¹═H, R²—R⁵ are not C₁₋₆ alkyl, e.g., CH₃;
when X═CH and R¹═H, R²—R⁵ are not Cl;
when X═CH and R¹═H, R² and R³ and R⁴ and R⁵ are not simultaneously bromo.

In one embodiment, provided herein is a compound selected from the group consisting of:

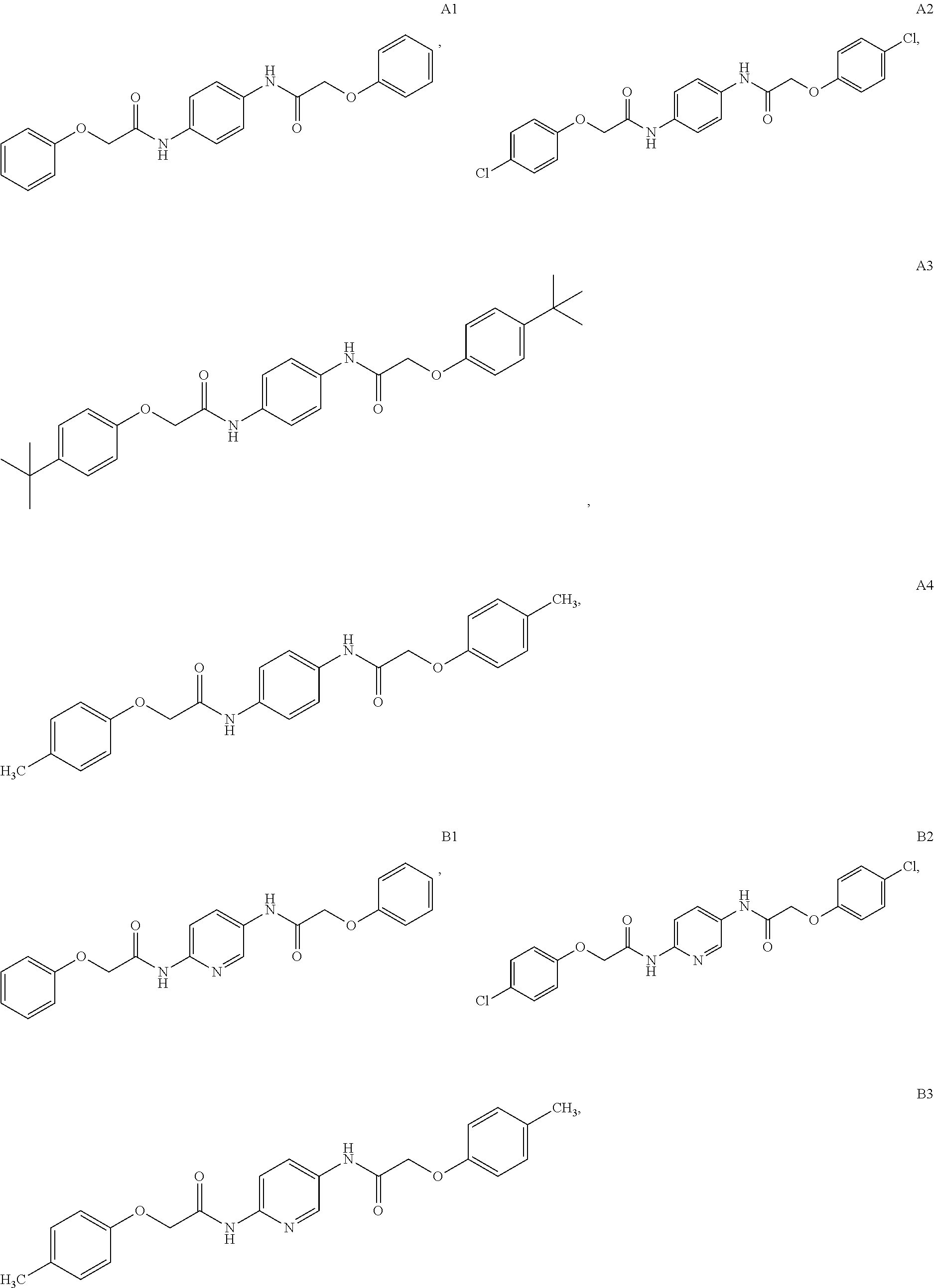

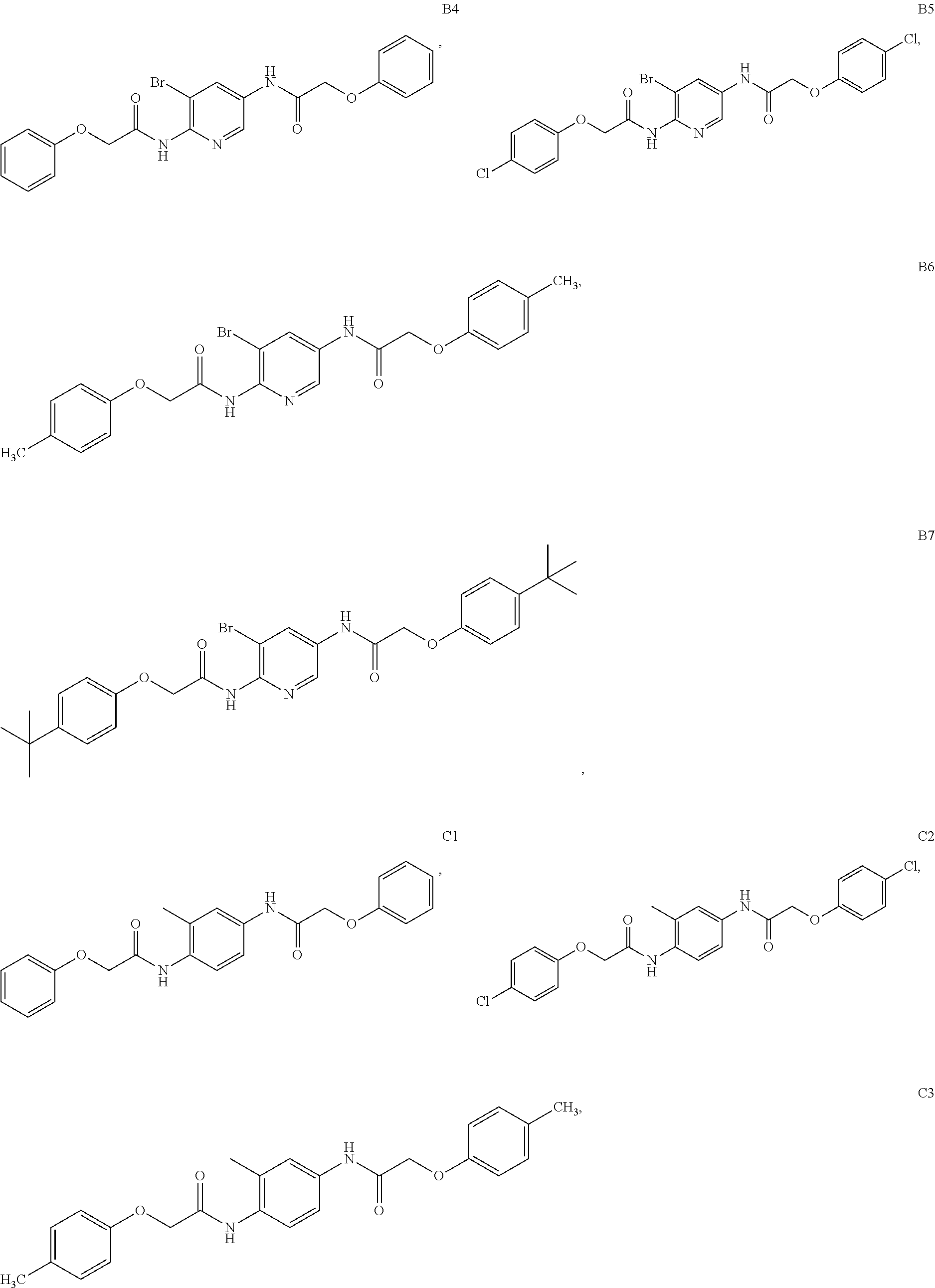

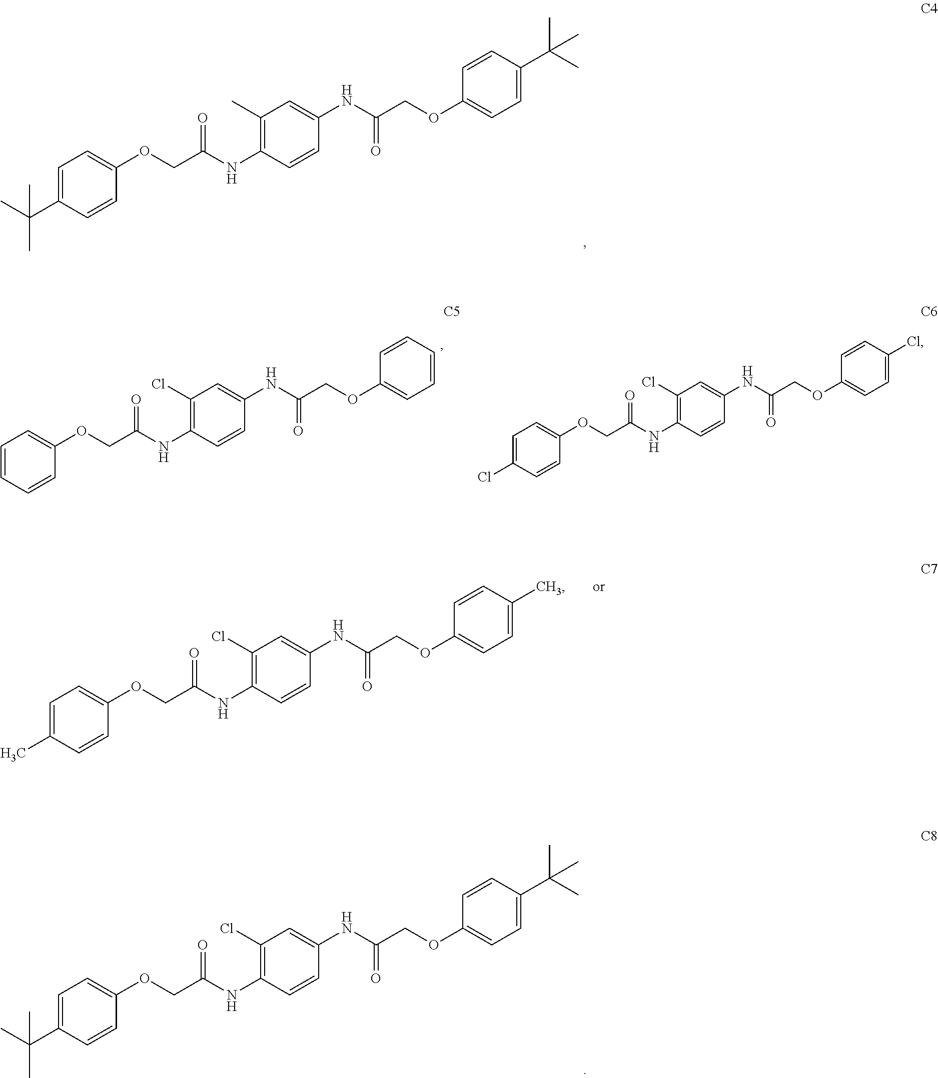

and pharmaceutically acceptable salts, solvates, and hydrates thereof.

Another embodiment is a pharmaceutical composition comprising a compound disclosed herein and a pharmaceutically acceptable carrier or diluent. In certain embodiments, the compounds provided herein show activity as modulators of the UPR. In certain embodiments, the compounds provided herein show activity as modulators of ER stress.

When a compound provided herein contains an acidic or basic moiety, it may also be provided as a pharmaceutically acceptable salt (See, Berge et al., J. Pharm. Sci. 1977, 66, 1-19; and “Handbook of Pharmaceutical Salts, Properties, and Use,” Stahl and Wermuth, Ed.; Wiley-VCH and VHCA, Zurich, 2002).

Suitable acids for use in the preparation of pharmaceutically acceptable salts include, but are not limited to, acetic acid, 2,2-dichloroacetic acid, acylated amino acids, adipic acid, alginic acid, ascorbic acid, L-aspartic acid, benzenesulfonic acid, benzoic acid, 4-acetamidobenzoic acid, boric acid, (+)-camphoric acid, camphorsulfonic acid, (+)-(1S)-camphor-10-sulfonic acid, capric acid, caproic acid, caprylic acid, cinnamic acid, citric acid, cyclamic acid, cyclohexanesulfamic acid, dodecylsulfuric acid, ethane-1,2-disulfonic acid, ethanesulfonic acid, 2-hydroxy-ethanesulfonic acid, formic acid, fumaric acid, galactaric acid, gentisic acid, glucoheptonic acid, D-gluconic acid, D-glucuronic acid, L-glutamic acid, α-oxoglutaric acid, glycolic acid, hippuric acid, hydrobromic acid, hydrochloric acid, hydroiodic acid, (+)-L-lactic acid, (±)-DL-lactic acid, lactobionic acid, lauric acid, maleic acid, (−)-L-malic acid, malonic acid, (±)-DL-mandelic acid, methanesulfonic acid, naphthalene-2-sulfonic acid, naphthalene-1,5-disulfonic acid, 1-hydroxy-2-naphthoic acid, nicotinic acid, nitric acid, oleic acid, orotic acid, oxalic acid, palmitic acid, pamoic acid, perchloric acid, phosphoric acid, L-pyroglutamic acid, saccharic acid, salicylic acid, 4-amino-salicylic acid, sebacic acid, stearic acid, succinic acid, sulfuric acid, tannic acid, (+)-L-tartaric acid, thiocyanic acid, p-toluenesulfonic acid, undecylenic acid, and valeric acid.

Suitable bases for use in the preparation of pharmaceutically acceptable salts, include, but are not limited to, inorganic bases, such as magnesium hydroxide, calcium hydroxide, potassium hydroxide, zinc hydroxide, or sodium hydroxide; and organic bases, such as primary, secondary, tertiary, and quaternary, aliphatic and aromatic amines, including, but not limited to, L-arginine, benethamine, benzathine, choline, deanol, diethanolamine, diethylamine, dimethylamine, dipropylamine, diisopropylamine, 2-(diethylamino)-ethanol, ethanolamine, ethylamine, ethylenediamine, isopropylamine, N-methyl-glucamine, hydrabamine, 1H-imidazole, L-lysine, morpholine, 4-(2-hydroxyethyl)-morpholine, methylamine, piperidine, piperazine, propylamine, pyrrolidine, 1-(2-hydroxyethyl)-pyrrolidine, pyridine, quinuclidine, quinoline, isoquinoline, secondary amines, triethanolamine, trimethylamine, triethylamine, N-methyl-D-glucamine, 2-amino-2-(hydroxymethyl)-1,3-propanediol, and tromethamine.

### Methods Of Synthesis

The compounds provided herein can be prepared, isolated, or obtained by any methods known to one of skill in the art, and the following examples are only representative and do not exclude other related procedures.

In one embodiment, for example, a compound of Formula I is prepared, as shown in Scheme 1 to form compound I.

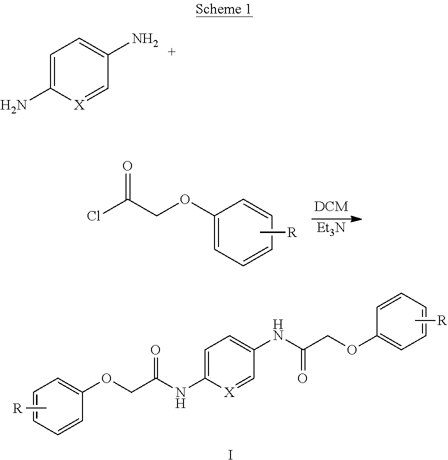

### Pharmaceutical Compositions

In one embodiment, provided herein is a pharmaceutical composition comprising the compound provided herein, including a compound of Formula I, or a pharmaceutically acceptable salt, solvate, or hydrate thereof; and a pharmaceutically acceptable excipient.

Pharmaceutical compositions of the present disclosure may be in solid forms (e.g., powders, suspensions, tablets, capsules, wafers, patches, and the like), liquid forms (e.g., solutions, suspensions, elixirs, syrups, sprays, and the like) and semi-solid forms (e.g., lotions, gels, emulsions, and the like). Powders may include amorphous powders, crystalline powders, and mixtures thereof. Tablets may include chewable tablets, non-chewable tablets, ingestible tablets, buccal tablets, troches, lozenges, suppositories, and the like. Capsules may include hard-shell capsules and soft-shell capsules in the form of troches, lozenges, suppositories, and the like. Solutions include aqueous solutions, non-aqueous solutions and mixed (aqueous/non-aqueous) solutions in the form of parenteral solutions, injectable solutions, infusible solutions, and the like.

The composition can be formulated to contain a daily dose or an appropriate portion of the daily dose in a dosage unit, which can be a single tablet or capsule or a liquid of a suitable volume.

In one embodiment, the solution is prepared from a water-soluble salt, such as hydrochloride. Generally, all compositions are prepared according to known methods in pharmaceutical chemistry. Capsules can be prepared by mixing the compound with a suitable carrier or diluent and filling an appropriate amount of the mixture into the capsule. Commonly used carriers and diluents include, but are not limited to, inert powdered substances, such as a variety of different starches, powdered cellulose, especially crystalline and microcrystalline cellulose, sugars like fructose, mannitol and sucrose, cereal flour, and similar edible powder.

Tablets can be prepared by direct compression, wet granulation, or dry granulation. The preparation usually adds diluent, binder, lubricant and disintegrant and the compound. Typical diluents include, for example, various types of starch, lactose, mannitol, kaolin, calcium phosphate or sulfate, inorganic salts (such as sodium chloride) and powdered sugar. Powdered cellulose derivatives are also useful. Typical tablet binders are the following substances, such as starch, gelatin, and sugar (such as lactose, fructose, glucose, etc.). Natural and synthetic gums are also suitable, including gum acacia, alginate, methylcellulose, polyvinylpyrrolidone, etc. Polyethylene glycol, ethylcellulose and wax can also act as binders.

Lubricants can be selected from such slippery solids such as talc, magnesium stearate and calcium stearate, stearic acid and hydrogenated vegetable oils. A tablet disintegrant swells when wet to break the tablet and release the compound. They include starch, clay, cellulose, algin and gum. More specifically, for example, corn and potato starch, methylcellulose, agar, bentonite, lignocellulose, powdered natural sponge, anion exchange resin, alginic acid, guar gum, citrus pomace, carboxymethylcellulose and sodium lauryl sulfate can be used. Tablets can be coated with sugar as a flavoring and sealing agent or coated with a film-forming protective agent to optimize the dissolution performance of the tablet. The composition can also be formulated into chewable tablets, for example, by adding some substances to the formulation, such as mannitol.

### Methods Of Treatment

In one embodiment, provided herein is a method for treating, preventing, or ameliorating one or more symptoms of a hearing loss condition, comprising administering to the subject a therapeutically effective amount of a compound provided herein, e.g., a compound of Formula I, or an enantiomer, a mixture of enantiomers, a mixture of two or more diastereomers,; or a pharmaceutically acceptable salt, solvate, or hydrate thereof. “Preventing” hearing loss means slowing the progression of hearing loss or reducing the likelihood of developing hearing loss in a subject at risk of developing hearing loss. Subjects at risk of developing hearing loss include elderly subjects (e.g., greater than sixty years of each), subjects with genetic pre-disposition towards hearing loss, subjects with acute sound exposure, subjects with sub-acute or chronic exposure to sound or subjects with drug exposure from, for example, aminoglycosides (e.g. tobramycin and amikacin), anti-cancer drugs (cisplatin) and loop diuretics (furosemide) and phosphodiesterase 5 inhibitors (tadalafil).

In yet another embodiment, provided herein is a method for treating, preventing, or ameliorating one or more symptoms of hearing loss in a subject, comprising administering to the subject a therapeutically effective amount of a compound provided herein, e.g., a compound of Formula I, or an enantiomer, a mixture of enantiomers, a mixture of two or more diastereomers; or a pharmaceutically acceptable salt, solvate, or hydrate thereof. In one embodiment, the diabetes is type 1. In one embodiment, the diabetes is type 2.

In yet another embodiment, provided herein is a method for treating age-related hearing loss in a subject, comprising administering to the subject a therapeutically effective amount of a compound provided herein, e.g., a compound of Formula I, or an enantiomer, a mixture of enantiomers, a mixture of two or more diastereomers; or a pharmaceutically acceptable salt, solvate, or hydrate thereof.

In yet another embodiment, provided herein is a method for treating noise-induced hearing loss in a subject, comprising administering to the subject a therapeutically effective amount of a compound provided herein, e.g., a compound of Formula I, or an enantiomer, a mixture of enantiomers, a mixture of two or more diastereomers; or a pharmaceutically acceptable salt, solvate, or hydrate thereof.

In yet another embodiment, provided herein is a method for treating genetic or inherited hearing loss in a subject, comprising administering to the subject a therapeutically effective amount of a compound provided herein, e.g., a compound of Formula I, or an enantiomer, a mixture of enantiomers, a mixture of two or more diastereomers; or a pharmaceutically acceptable salt, solvate, or hydrate thereof.

In yet another embodiment, provided herein is a method for treating ototoxicity-induced hearing loss in a subject, comprising administering to the subject a therapeutically effective amount of a compound provided herein, e.g., a compound of Formula I, or an enantiomer, a mixture of enantiomers, a mixture of two or more diastereomers; or a pharmaceutically acceptable salt, solvate, or hydrate thereof.

In yet another embodiment, provided herein is a method for treating disease-induced hearing loss in a subject, comprising administering to the subject a therapeutically effective amount of a compound provided herein, e.g., a compound of Formula I, or an enantiomer, a mixture of enantiomers, a mixture of two or more diastereomers; or a pharmaceutically acceptable salt, solvate, or hydrate thereof.

In yet another embodiment, provided herein is a method for treating trauma-induced hearing loss in a subject, comprising administering to the subject a therapeutically effective amount of a compound provided herein, e.g., a compound of Formula I, or an enantiomer, a mixture of enantiomers, a mixture of two or more diastereomers; or a pharmaceutically acceptable salt, solvate, or hydrate thereof.

In yet another embodiment, provided herein is a method for treating cochlear synaptopathy in a subject, comprising administering to the subject a therapeutically effective amount of a compound provided herein, e.g., a compound of Formula I, or an enantiomer, a mixture of enantiomers, a mixture of two or more diastereomers; or a pharmaceutically acceptable salt, solvate, or hydrate thereof.

In certain embodiments, the subject is a mammal. In certain embodiments, the subject is a human. In certain embodiments, the subject is a primate other than a human, a farm animal such as cattle, a sport animal, or a pet such as a horse, dog, or cat.

In the treatment, prevention, or amelioration of one or more symptoms of the disorders, diseases, or conditions described herein, an appropriate dosage level, i.e., a therapeutically effective amount, generally is ranging from about 0.001 to 1 gram

It will be understood, however, that the specific dose level and frequency of dosage for any particular patient can be varied and will depend upon a variety of factors including the activity of the specific compound employed, the metabolic stability and length of action of that compound, the age, body weight, general health, sex, diet, mode and time of administration, rate of excretion, drug combination, the severity of the particular condition, and the host undergoing therapy.

The compound provided herein, e.g., a compound of Formula I,; or a pharmaceutically acceptable salt, solvate, or hydrate thereof; can also be combined or used in combination with other agents or therapies useful in the treatment, prevention, or amelioration of one or more symptoms of the disorders, diseases, or conditions for which the compounds provided herein are useful.

Suitable other therapeutic agents can also include, but are not limited to, (1) alpha-adrenergic agents; (2) antiarrhythmic agents; (3) anti-atherosclerotic agents, such as ACAT inhibitors; (4) antibiotics, such as anthracyclines, bleomycins, mitomycin, dactinomycin, and plicamycin; (5) anticancer agents and cytotoxic agents, e.g., alkylating agents, such as nitrogen mustards, alkyl sulfonates, nitrosoureas, ethylenimines, and triazenes; (6) anticoagulants, such as acenocoumarol, argatroban, bivalirudin, lepirudin, fondaparinux, heparin, phenindione, warfarin, and ximelagatran; (7) anti-diabetic agents, such as biguanides (e.g., metformin), glucosidase inhibitors (e.g., acarbose), insulins, meglitinides (e.g., repaglinide), sulfonylureas (e.g., glimepiride, glyburide, and glipizide), thiozolidinediones (e.g., troglitazone, rosiglitazone, and pioglitazone), and PPAR-gamma agonists; (8) antifungal agents, such as amorolfine, amphotericin B, anidulafungin, bifonazole, butenafine, butoconazole, caspofungin, ciclopirox, clotrimazole, econazole, fenticonazole, filipin, fluconazole, isoconazole, itraconazole, ketoconazole, micafungin, miconazole, naftifine, natamycin, nystatin, oxyconazole, ravuconazole, posaconazole, rimocidin, sertaconazole, sulconazole, terbinafine, terconazole, tioconazole, and voriconazole; (9) antiinflammatories, e.g., non-steroidal anti-inflammatory agents, such as aceclofenac, acemetacin, amoxiprin, aspirin, azapropazone, benorilate, bromfenac, carprofen, celecoxib, choline magnesium salicylate, diclofenac, diflunisal, etodolac, etoricoxib, faislamine, fenbufen, fenoprofen, flurbiprofen, ibuprofen, indometacin, ketoprofen, ketorolac, lornoxicam, loxoprofen, lumiracoxib, meclofenamic acid, mefenamic acid, meloxicam, metamizole, methyl salicylate, magnesium salicylate, nabumetone, naproxen, nimesulide, oxyphenbutazone, parecoxib, phenylbutazone, piroxicam, salicyl salicylate, sulindac, sulfinpyrazone, suprofen, tenoxicam, tiaprofenic acid, and tolmetin; (10) antimetabolites, such as folate antagonists, purine analogues, and pyrimidine analogues; (11) anti-platelet agents, such as GPIIb/IIIa blockers (e.g., abciximab, eptifibatide, and tirofiban), P2Y (AC) antagonists (e.g., clopidogrel, ticlopidine and CS-747), cilostazol, dipyridamole, and aspirin; (12) antiproliferatives, such as methotrexate, FK506 (tacrolimus), and mycophenolate mofetil; (13) anti-TNF antibodies or soluble TNF receptor, such as etanercept, rapamycin, and leflunimide; (14) aP2 inhibitors; (15) beta-adrenergic agents, such as carvedilol and metoprolol; (16) bile acid sequestrants, such as questran; (17) calcium channel blockers, such as amlodipine besylate; (18) chemotherapeutic agents; (19) cyclooxygenase-2 (COX-2) inhibitors, such as celecoxib and rofecoxib; (20) cyclosporins; (21) cytotoxic drugs, such as azathioprine and cyclophosphamide; (22) diuretics, such as chlorothiazide, hydrochlorothiazide, flumethiazide, hydroflumethiazide, bendroflumethiazide, methylchlorothiazide, trichloromethiazide, polythiazide, benzothiazide, ethacrynic acid, ticrynafen, chlorthalidone, furosenide, muzolimine, bumetanide, triamterene, amiloride, and spironolactone; (23) endothelin converting enzyme (ECE) inhibitors, such as phosphoramidon; (24) enzymes, such as L-asparaginase; (25) Factor VIIa Inhibitors and Factor Xa Inhibitors; (26) farnesyl-protein transferase inhibitors; (27) fibrates; (28) growth factor inhibitors, such as modulators of PDGF activity; (29) growth hormone secretagogues; (30) HMG CoA reductase inhibitors, such as pravastatin, lovastatin, atorvastatin, simvastatin, NK-104 (a.k.a. itavastatin, nisvastatin, or nisbastatin), and ZD-4522 (also known as rosuvastatin, atavastatin, or visastatin); neutral endopeptidase (NEP) inhibitors; (31) hormonal agents, such as glucocorticoids (e.g., cortisone), estrogens/antiestrogens, androgens/antiandrogens, progestins, and luteinizing hormone-releasing hormone antagonists, and octreotide acetate; (32) immunosuppressants; (33) mineralocorticoid receptor antagonists, such as spironolactone and eplerenone; (34) microtubule-disruptor agents, such as ecteinascidins; (35) microtubule-stabilizing agents, such as pacitaxel, docetaxel, and epothilones A-F; (36) MTP Inhibitors; (37) niacin; (38) phosphodiesterase inhibitors, such as PDE III inhibitors (e.g., cilostazol) and PDE V inhibitors (e.g., sildenafil, tadalafil, and vardenafil); (39) plant-derived products, such as vinca alkaloids, epipodophyllotoxins, and taxanes; (40) platelet activating factor (PAF) antagonists; (41) platinum coordination complexes, such as cisplatin, satraplatin, and carboplatin; (42) potassium channel openers; (43) prenyl-protein transferase inhibitors; (44) protein tyrosine kinase inhibitors; (45) renin inhibitors; (46) squalene synthetase inhibitors; (47) steroids, such as aldosterone, beclometasone, betamethasone, deoxycorticosterone acetate, fludrocortisone, hydrocortisone (cortisol), prednisolone, prednisone, methylprednisolone, dexamethasone, and triamcinolone; (48) TNF-alpha inhibitors, such as tenidap; (49) thrombin inhibitors, such as hirudin; (50) thrombolytic agents, such as anistreplase, reteplase, tenecteplase, tissue plasminogen activator (tPA), recombinant tPA, streptokinase, urokinase, prourokinase, and anisoylated plasminogen streptokinase activator complex (APSAC); (51) thromboxane receptor antagonists, such as ifetroban; (52) topoisomerase inhibitors; (53) vasopeptidase inhibitors (dual NEP-ACE inhibitors), such as omapatrilat and gemopatrilat; and (54) other miscellaneous agents, such as, hydroxyurea, procarbazine, mitotane, hexamethylmelamine, and gold compounds.

In certain embodiments, the other therapies that may be used in combination with the compounds provided herein include, but are not limited to, surgery, endocrine therapy, biologic response modifiers (e.g., interferons, interleukins, and tumor necrosis factor (TNF)), hyperthermia and cryotherapy, and agents to attenuate any adverse effects (e.g., antiemetics).

Such other agents, or drugs, can be administered, by a route and in an amount commonly used therefor, simultaneously or sequentially with the compound provided herein, e.g., a compound of Formula I,; or a pharmaceutically acceptable salt, solvate, hydrate, or prodrug thereof. When a compound provided herein is used contemporaneously with one or more other drugs, a pharmaceutical composition containing such other drugs in addition to the compound provided herein can be utilized, but is not required. Accordingly, the pharmaceutical compositions provided herein include those that also contain one or more other active ingredients or therapeutic agents, in addition to a compound provided herein.

### EXAMPLES

The disclosure will be further understood by the following non-limiting examples.

As used herein, the symbols and conventions used in these processes, schemes and examples, regardless of whether a particular abbreviation is specifically defined, are consistent with those used in the contemporary scientific literature, for example, the Journal of the American Chemical Society or the Journal of Biological Chemistry. Specifically, but without limitation, the following abbreviations may be used in the examples and throughout the specification: g (grams); mg (milligrams); mL (milliliters); μL (microliters); M (molar); mM (millimolar); μM (micromolar); mol (moles); mmol (millimoles); hr or hrs (hour or hours); and min (minutes).

For all of the following examples, standard procedures and methods known to those skilled in the art can be utilized. Unless otherwise indicated, all temperatures are expressed in ° C. (degrees Centigrade). All procedures are conducted at room temperature unless otherwise noted.

### Biological Assays

### ER Stress Induction in HEK Cells Treated With Compounds

HEK 293 cells were cultured in DMEM with 10% FBS, Pen/Strep in 5% CO2, at 37° C. in incubator. Cells were split and seeded at approximately 2×104 cells in each well of 96-well plate one day before treatment. Cells were grown in good shape and passaged one or two times before testing.

Cells were treated with 0.5 μM thapsigargin and 0.1 to 5.0 μM of each drug for 2 hours. The cells were then harvested, pelleted and treated to ready for testing mRNA abundance for specific UPR genes from cells in the PCR reaction using the TaqMan™ Gene Expression Cells-to-CT™ Kit (Thermo Fisher). Prescreened primers were ordered from Bio-Rad. The gene tested is CHOP, (the C/EBP Homologous Protein). These markers were compared against GAPDH. The qPCR system we used was CFX 384 Real time (Bio-Rad). Samples were measured in triplicates and relative quantification, by the ΔΔCt-method, using GAPDH as reference. The biological results are summarized in Table 1 where A represents a greater than 50% reduction in CHOP signal in compound-treated versus untreated cells, B represents a 20% to 50% reduction in CHOP signal, and C represents a 1% to 20% reduction in CHOP signal.

TABLE 1

| Cmpd. | CHOP reduction | Cmpd. | CHOP reduction |
|---|---|---|---|
| A1 | A | A2 | B |
| A3 | C | A4 | C |
| B1 | C | B2 | A |
| B3 | B | B4 | C |
| B5 | C | B6 | C |
| B7 | B | C1 | B |
| C2 | A | C4 | C |
| C5 | B | C6 | A |
| C7 | C | C8 | B |

### Compound Synthesis

2-Phenoxy-N-[4-(2-phenoxyacetylamino) phenyl]acetamide A1

p-Phenylenediamine (1.0 eq.), phenoxyacetyl chloride (1.05 eq.), and triethylamine (1.2 eq.) were dissolved in dichloromethane and the reaction mixture was stirred at room temperature for 15 hrs. The mixture was then diluted with water and extracted with 2 volumes of dichloromethane. The organic layers were collected, and the solvent was removed by rotary evaporation. The residue was purified by preparative reverse-phase HPLC using a water-acetonitrile gradient to afford the desired compound A1. ESI-LCMS: m/z 377 [M+H]⁺.

2-(4-Chlorophenoxy)-N-[4-[[2-(4-chlorophenoxy)acetyl]amino]phenyl]acetamide A2

p-Phenylenediamine (1.0 eq.), 4-chlorophenoxyacetyl chloride (1.05 eq.), and triethylamine (1.2 eq.) were dissolved in dichloromethane and the reaction mixture was stirred at room temperature for 15 hrs. The mixture was then diluted with water and extracted with 2 volumes of dichloromethane. The organic layers were collected, and the solvent was removed by rotary evaporation. The residue was purified by preparative reverse-phase HPLC using a water-acetonitrile gradient to afford the desired compound A2. ESI-LCMS: m/z 446 [M+H]⁺.

N,N′-Benzene-1,4-diylbis [2-(4-tert-butylphenoxy) acetamide]A3

p-Phenylenediamine (1.0 eq.), 4-tert-butylphenoxyacetyl chloride (1.05 eq.), and triethylamine (1.2 eq.) were dissolved in dichloromethane and the reaction mixture was stirred at room temperature for 15 hrs. The mixture was then diluted with water and extracted with 2 volumes of dichloromethane. The organic layers were collected, and the solvent was removed by rotary evaporation. The residue was purified by preparative reverse-phase HPLC using a water-acetonitrile gradient to afford the desired compound A3. ESI-LCMS: m/z 489 [M+H]⁺.

N,N′-Benzene-1,4-diylbis [2-(4-methylphenoxy) acetamide]A4

p-Phenylenediamine (1.0 eq.), 4-methylphenoxyacetyl chloride (1.05 eq.), and triethylamine (1.2 eq.) were dissolved in dichloromethane and the reaction mixture was stirred at room temperature for 15 hrs. The mixture was then diluted with water and extracted with 2 volumes of dichloromethane. The organic layers were collected, and the solvent was removed by rotary evaporation. The residue was purified by preparative reverse-phase HPLC using a water-acetonitrile gradient to afford the desired compound A4. ESI-LCMS: m/z 405 [M+H]⁺.

2-Phenoxy-N-[6-[[2-phenoxyacetyl]amino]pyridin-3-yl]acetamide B1

2,5-Diaminopyridine (1.0 eq.), phenoxyacetyl chloride (1.05 eq.), and triethylamine (1.2 eq.) were dissolved in dichloromethane and the reaction mixture was stirred at room temperature for 15 hrs. The mixture was then diluted with water and extracted with 2 volumes of dichloromethane. The organic layers were collected, and the solvent was removed by rotary evaporation. The residue was purified by preparative reverse-phase HPLC using a water-acetonitrile gradient to afford the desired compound B1. ESI-LCMS: m/z 378 [M+H]⁺.

2-(4-Chlorophenoxy)-N-[6-[[2-(4-chlorophenoxy)acetyl]amino]pyridin-3-yl]acetamide B2

2,5-Diaminopyridine (1.0 eq.), 4-chlorophenoxyacetyl chloride (1.05 eq.), and triethylamine (1.2 eq.) were dissolved in dichloromethane and the reaction mixture was stirred at room temperature for 15 hrs. The mixture was then diluted with water and extracted with 2 volumes of dichloromethane. The organic layers were collected, and the solvent was removed by rotary evaporation. The residue was purified by preparative reverse-phase HPLC using a water-acetonitrile gradient to afford the desired compound B2. ESI-LCMS: m/z 447 [M+H]⁺.

2-(4-Methylphenoxy)-N-[6-[[2-(4-methylphenoxy)acetyl]amino]pyridin-3-yl]acetamide B3

2,5-Diaminopyridine (1.0 eq.), 4-methylphenoxyacetyl chloride (1.05 eq.), and triethylamine (1.2 eq.) were dissolved in dichloromethane and the reaction mixture was stirred at room temperature for 15 hrs. The mixture was then diluted with water and extracted with 2 volumes of dichloromethane. The organic layers were collected, and the solvent was removed by rotary evaporation. The residue was purified by preparative reverse-phase HPLC using a water-acetonitrile gradient to afford the desired compound B3. ESI-LCMS: m/z 406 [M+H]⁺.

2-Phenoxy-N-[5-bromo-6-[[2-phenoxyacetyl]amino]pyridin-3-yl]acetamide B4

3-Bromo-2,5-diaminopyridine (1.0 eq.), phenoxyacetyl chloride (1.05 eq.), and triethylamine (1.2 eq.) were dissolved in dichloromethane and the reaction mixture was stirred at room temperature for 15 hrs. The mixture was then diluted with water and extracted with 2 volumes of dichloromethane. The organic layers were collected, and the solvent was removed by rotary evaporation. The residue was purified by preparative reverse-phase HPLC using a water-acetonitrile gradient to afford the desired compound B4. ESI-LCMS: m/z 457 [M+H]⁺.

2-(4-Chlorophenoxy)-N-[5-bromo-6-[[2-(4-chlorophenoxy)acetyl]amino]pyridin-3-yl]acetamide B5

3-Bromo-2,5-diaminopyridine (1.0 eq.), 4-chlorophenoxyacetyl chloride (1.05 eq.), and triethylamine (1.2 eq.) were dissolved in dichloromethane and the reaction mixture was stirred at room temperature for 15 hrs. The mixture was then diluted with water and extracted with 2 volumes of dichloromethane. The organic layers were collected, and the solvent was removed by rotary evaporation. The residue was purified by preparative reverse-phase HPLC using a water-acetonitrile gradient to afford the desired compound B5. ESI-LCMS: m/z 526 [M+H]⁺.

2-(4-Methylphenoxy)-N-[5-bromo-6-[[2-(4-methylphenoxy)acetyl]amino]pyridin-3-yl]acetamide B6

3-Bromo-2,5-diaminopyridine (1.0 eq.), 4-methylphenoxyacetyl chloride (1.05 eq.), and triethylamine (1.2 eq.) were dissolved in dichloromethane and the reaction mixture was stirred at room temperature for 15 hrs. The mixture was then diluted with water and extracted with 2 volumes of dichloromethane. The organic layers were collected, and the solvent was removed by rotary evaporation. The residue was purified by preparative reverse-phase HPLC using a water-acetonitrile gradient to afford the desired compound B6. ESI-LCMS: m/z 485 [M+H]⁺.

2-(4-tert-Butylphenoxy)-N-[5-bromo-6-[[2-(4-tert-butylphenoxy)acetyl]amino]pyridin-3-yl]acetamide B7

3-Bromo-2,5-diaminopyridine (1.0 eq.), 4-tert-butylphenoxyacetyl chloride (1.05 eq.), and triethylamine (1.2 eq.) were dissolved in dichloromethane and the reaction mixture was stirred at room temperature for 15 hrs. The mixture was then diluted with water and extracted with 2 volumes of dichloromethane. The organic layers were collected, and the solvent was removed by rotary evaporation. The residue was purified by preparative reverse-phase HPLC using a water-acetonitrile gradient to afford the desired compound B7. ESI-LCMS: m/z 569 [M+H]⁺.

2-Phenoxy-N-[2-methyl-4-(2-phenoxyacetylamino)phenyl]acetamide C1

2,5-Diaminotoluene (1.0 eq.), phenoxyacetyl chloride (1.05 eq.), and triethylamine (1.2 eq.) were dissolved in dichloromethane and the reaction mixture was stirred at room temperature for 15 hrs. The mixture was then diluted with water and extracted with 2 volumes of dichloromethane. The organic layers were collected, and the solvent was removed by rotary evaporation. The residue was purified by preparative reverse-phase HPLC using a water-acetonitrile gradient to afford the desired compound C1. ESI-LCMS: m/z 391 [M+H]⁺.

2-(4-Chlorophenoxy)-N-[2-methyl-4-[[2-(4-chlorophenoxy)acetyl]amino]phenyl]acetamide C2

2,5-Diaminotoluene (1.0 eq.), 4-chlorophenoxyacetyl chloride (1.05 eq.), and triethylamine (1.2 eq.) were dissolved in dichloromethane and the reaction mixture was stirred at room temperature for 15 hrs. The mixture was then diluted with water and extracted with 2 volumes of dichloromethane. The organic layers were collected, and the solvent was removed by rotary evaporation. The residue was purified by preparative reverse-phase HPLC using a water-acetonitrile gradient to afford the desired compound C2. ESI-LCMS: m/z 460 [M+H]⁺.

N,N′-Toluene-1,4-diylbis[2-(4-methylphenoxy)acetamide]C3

2,5-Diaminotoluene (1.0 eq.), 4-methylphenoxyacetyl chloride (1.05 eq.), and triethylamine (1.2 eq.) were dissolved in dichloromethane and the reaction mixture was stirred at room temperature for 15 hrs. The mixture was then diluted with water and extracted with 2 volumes of dichloromethane. The organic layers were collected, and the solvent was removed by rotary evaporation. The residue was purified by preparative reverse-phase HPLC using a water-acetonitrile gradient to afford the desired compound C3. ESI-LCMS: m/z 419 [M+H]⁺.

N,N′-Toluene-1,4-diylbis [2-(4-tert-butylphenoxy)acetamide]C4

2,5-Diaminotoluene (1.0 eq.), 4-tert-butylphenoxyacetyl chloride (1.05 eq.), and triethylamine (1.2 eq.) were dissolved in dichloromethane and the reaction mixture was stirred at room temperature for 15 hrs. The mixture was then diluted with water and extracted with 2 volumes of dichloromethane. The organic layers were collected, and the solvent was removed by rotary evaporation. The residue was purified by preparative reverse-phase HPLC using a water-acetonitrile gradient to afford the desired compound C4. ESI-LCMS: m/z 503 [M+H]⁺.

2-Phenoxy-N-[2-chloro-4-(2-phenoxyacetylamino)phenyl]acetamide C5

2-Chloro-1,4-phenylenediamine (1.0 eq.), phenoxyacetyl chloride (1.05 eq.), and triethylamine (1.2 eq.) were dissolved in dichloromethane and the reaction mixture was stirred at room temperature for 15 hrs. The mixture was then diluted with water and extracted with 2 volumes of dichloromethane. The organic layers were collected, and the solvent was removed by rotary evaporation. The residue was purified by preparative reverse-phase HPLC using a water-acetonitrile gradient to afford the desired compound C5. ESI-LCMS: m/z 411 [M+H]⁺.

2-(4-Chlorophenoxy)-N-[2-chloro-4-[[2-(4-chlorophenoxy)acetyl]amino]phenyl]acetamide C6

2-Chloro-1,4-phenylenediamine (1.0 eq.), 4-chlorophenoxyacetyl chloride (1.05 eq.), and triethylamine (1.2 eq.) were dissolved in dichloromethane and the reaction mixture was stirred at room temperature for 15 hrs. The mixture was then diluted with water and extracted with 2 volumes of dichloromethane. The organic layers were collected, and the solvent was removed by rotary evaporation. The residue was purified by preparative reverse-phase HPLC using a water-acetonitrile gradient to afford the desired compound C6. ESI-LCMS: m/z 480 [M+H]⁺.

N,N′-2-Chlorobenzene-1,4-diylbis[2-(4-methylphenoxy)acetamide]C7

2-Chloro-1,4-phenylenediamine (1.0 eq.), 4-methylphenoxyacetyl chloride (1.05 eq.), and triethylamine (1.2 eq.) were dissolved in dichloromethane and the reaction mixture was stirred at room temperature for 15 hrs. The mixture was then diluted with water and extracted with 2 volumes of dichloromethane. The organic layers were collected, and the solvent was removed by rotary evaporation. The residue was purified by preparative reverse-phase HPLC using a water-acetonitrile gradient to afford the desired compound C7. ESI-LCMS: m/z 439 [M+H]⁺.

N,N′-2-Chlorobenzene-1,4-diylbis[2-(4-tert-butylphenoxy)acetamide]C8

2-Chloro-1,4-phenylenediamine (1.0 eq.), 4-tert-butylphenoxyacetyl chloride (1.05 eq.), and triethylamine (1.2 eq.) were dissolved in dichloromethane and the reaction mixture was stirred at room temperature for 15 hrs. The mixture was then diluted with water and extracted with 2 volumes of dichloromethane. The organic layers were collected, and the solvent was removed by rotary evaporation. The residue was purified by preparative reverse-phase HPLC using a water-acetonitrile gradient to afford the desired compound C8. ESI-LCMS: m/z 524 [M+H]⁺.

The examples set forth above are provided to give those of ordinary skill in the art with a complete disclosure and description of how to make and use the claimed embodiments, and are not intended to limit the scope of what is disclosed herein. Modifications that are obvious to persons of skill in the art are intended to be within the scope of the following claims. All publications, patents, and patent applications cited in this specification are incorporated herein by reference as if each such publication, patent or patent application were specifically and individually indicated to be incorporated herein by reference.

What is claimed:

1. A method for treating hearing loss in a subject comprising administering to the subject a therapeutically effective amount of a compound of Formula I: 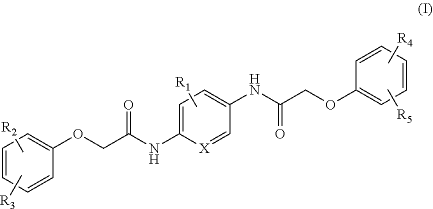
or a pharmaceutically acceptable salt, solvate, or hydrate thereof; wherein:
R¹-R⁵ are independently selected from hydrogen, cyano, nitro, or halo; C₁₋₆ unsubstituted alkyl, C₂₋₆ unsubstituted alkenyl, or C₂₋₆ unsubstituted alkynyl; —CF₃, —O—(C₁-C₄ alkyl), —O—CF₃, —OH, —NH(C₁-C₄ alkyl)₂ and —N(C₁-C₄ alkyl)₂; and
X is N or CH.

2. The method according to claim 1 comprising administering to the subject a therapeutically effective amount of a compound of Formula II: 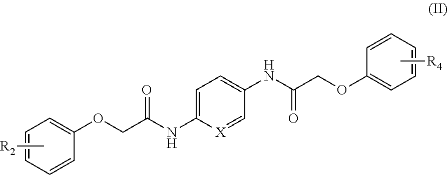
or a pharmaceutically acceptable salt, solvate, or hydrate thereof; wherein:
R¹ and R² are independently selected from hydrogen, cyano, nitro, or halo; C₁₋₆ unsubstituted alkyl, C₂₋₆ unsubstituted alkenyl, or C₂₋₆ unsubstituted alkynyl; —CF₃, —O—(C₁-C₄ alkyl), —O—CF₃, —OH, —NH(C₁-C₄ alkyl)₂ and —N(C₁-C₄ alkyl)₂; and
X is N or CH.

3. The method according to claim 1, wherein the compound is selected from the group consisting of: 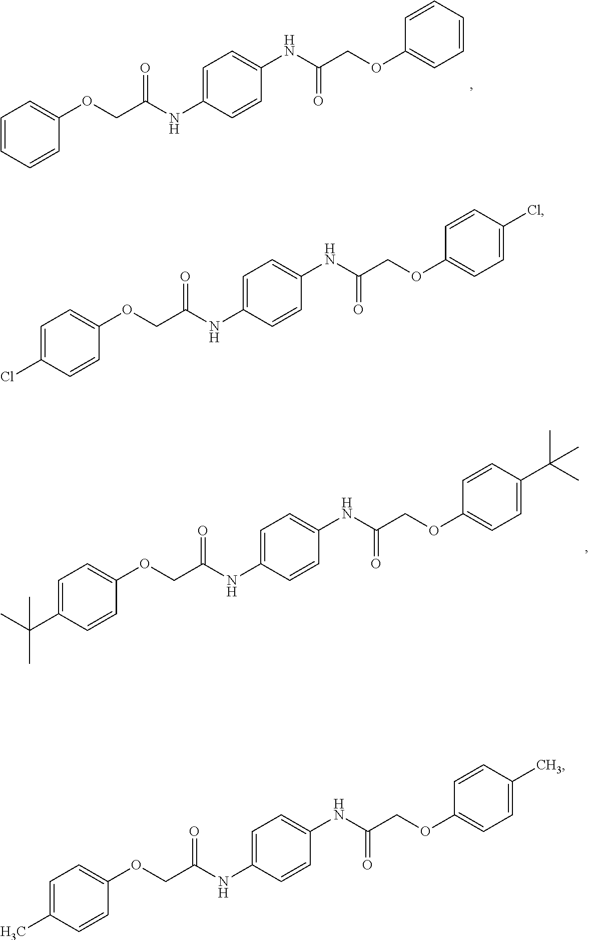 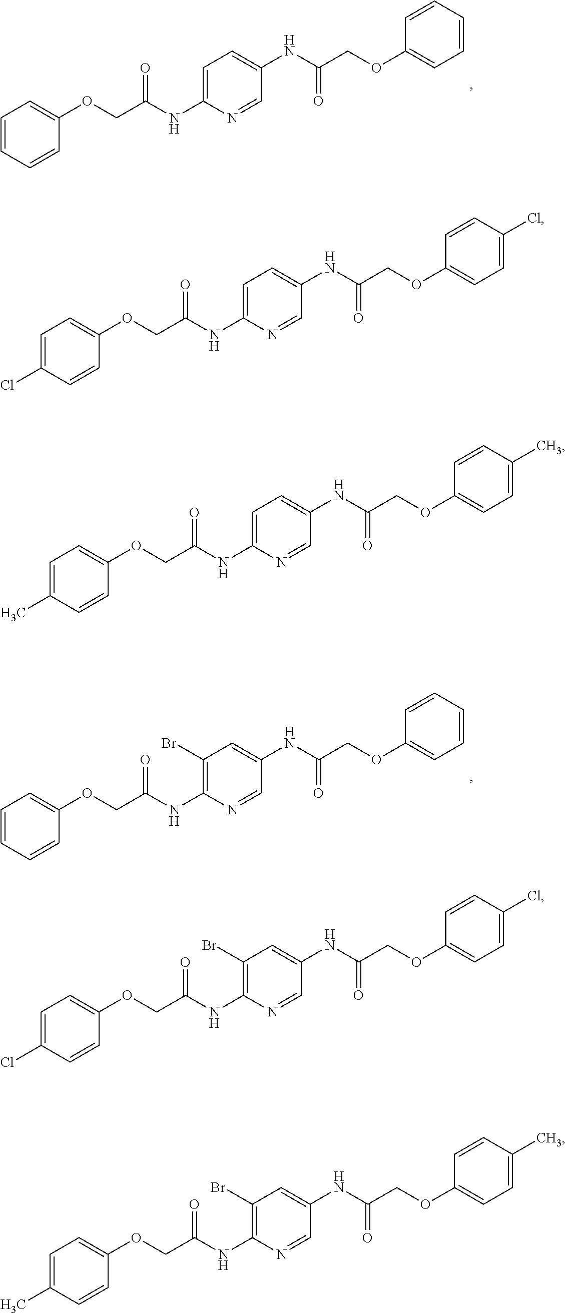 [SMILES: O=C(COc1ccccc1)Nc1ccc(NC(=O)COc2ccccc2)nc1 ; O=C(COc1ccc(Cl)cc1)Nc1ccc(NC(=O)COc2ccc(Cl)cc2)nc1 ; Cc1ccc(OCC(=O)Nc2ccc(NC(=O)COc3ccc(C)cc3)nc2)cc1 ; O=C(COc1ccccc1)Nc1cnc(NC(=O)COc2ccccc2)c(Br)c1 ; O=C(COc1ccc(Cl)cc1)Nc1cnc(NC(=O)COc2ccc(Cl)cc2)c(Br)c1 ; Cc1ccc(OCC(=O)Nc2cnc(NC(=O)COc3ccc(C)cc3)c(Br)c2)cc1] 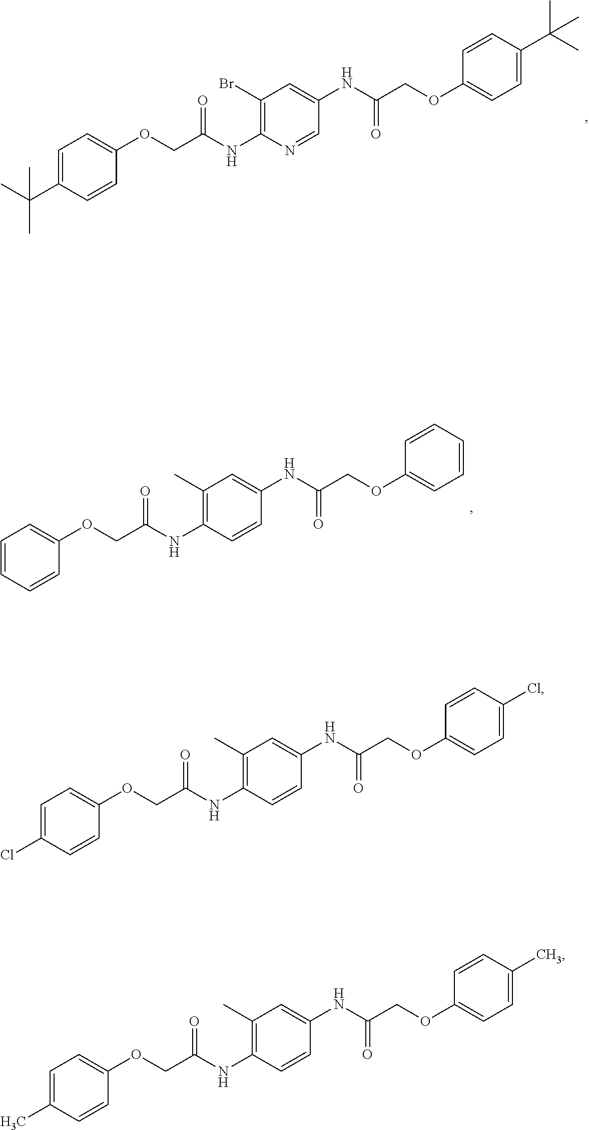 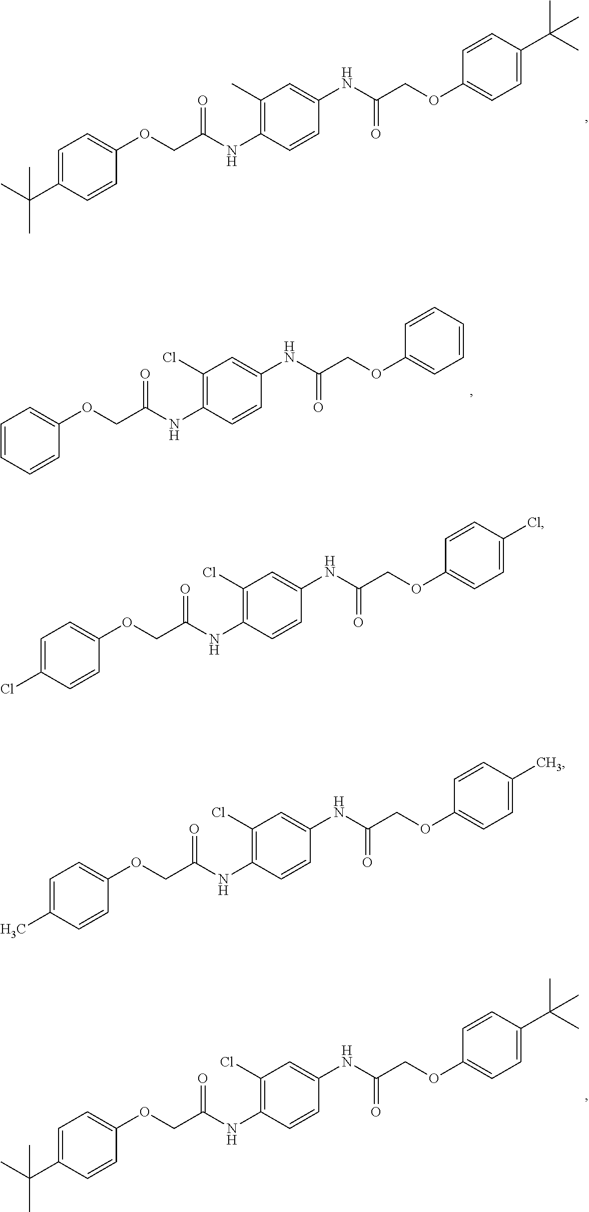
or a pharmaceutically acceptable salt, solvate, or hydrate thereof.

4. The method according to claim 1, wherein the compound is selected from the group consisting of: 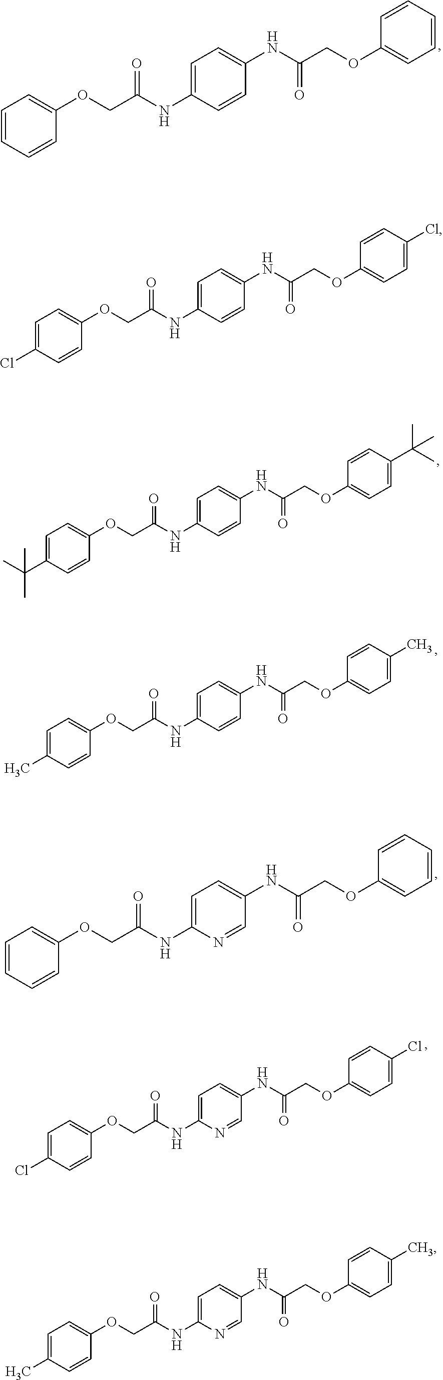
or a pharmaceutically acceptable salt thereof, solvate, or hydrate thereof.

5. The method of claims 1-4 wherein the hearing loss is age-related.

6. The method of claims 1-4 wherein the hearing loss is noise induced.

7. The method of claims 1-4 wherein the hearing loss is genetic or inherited.

8. The method of claims 1-4 wherein the hearing loss is due to ototoxicity.

9. The method of claims 1-4 wherein the hearing loss is due to disease.

10. The method of claims 1-4 wherein the hearing loss is due to trauma.

11. The method of claims 1-4 wherein the hearing loss is due to cochlear synaptopathy.
